(19) United States
(12) Patent Application Publication
(10) Pub. No.: US 2004/0185473 A1
(43) Pub. Date: Sep. 23, 2004

(54) Releasable polymer arrays

(75) Inventors: Andrea Cuppoletti, Livermore, CA (US); Glenn H. McGall, San Jose, CA (US)

Correspondence Address: AFFYMETRIX, INC, ATTN: CHIEF IP COUNSEL, LEGAL DEPT., 3380 CENTRAL EXPRESSWAY, SANTA CLARA, CA, 95051, US

(73) Assignee: Affymetrix, INC., Santa Clara, CA (US)

(21) Appl. No.: 10/738,381

(22) Filed: Dec. 16, 2003

Related U.S. Application Data
(60) Provisional application No. 60/434,144, filed on Dec. 17, 2002.

(51) Int. Cl. C12Q 1/68; C12P 19/34; C12M 1/34

(52) U.S. Cl. 435/6; 435/91.2; 435/287.2

(57) ABSTRACT

Methods are provided for fabricating an array of polymers wherein the polymers may be released from the surface of the array by activation of a releasable group. Also provided are methods for fabricating an array of polymers wherein the polymers can be released from the surface of the array by activation of a releasable group. Arrays of nucleic acids wherein a nucleic acid probe may be released from the array by activation of a releasable groups and methods for fabrication of such arrays are also disclosed.

### PRIORITY CLAIM

This application claims priority of U.S. Provisional Application Serial No. 60/434,144 filed on Dec. 17, 2002, which is incorporated herein referenced in its entirety.

### RELATED APPLICATIONS

This application is related to U.S. application Ser. No. 10/272,155 filed on Oct. 14, 2002, which is incorporated herein referenced in its entirety.

### FIELD OF THE INVENTION

The present invention relates generally to the field of polymer arrays. More specifically, the present invention relates to the release of polymers from an array using releasable groups.

### BACKGROUND OF THE INVENTION

U.S. Pat. No. 5,424,186 to Fodor, et al., describes a technique for, among other things, forming and using high density arrays of probes comprising molecules such as oligonucleotide, RNA, peptides, polysaccharides, and other materials. Arrays of oligonucleotides or peptides, for example, are formed on the surface by sequentially removing a photo-removable group from a surface, coupling a monomer to the exposed region of the surface, and repeating the process. Nucleic acid probe arrays synthesized in this manner, such as Affymetrix GeneChip® probe arrays from Affymetrix, Inc. of Santa Clara, Calif. have been used to generate unprecedented amounts of information about biological systems. Analysis of these data may lead to the development of new drugs and new diagnostic tools.

A typical step in the process of synthesizing these probe arrays is to design a mask that will define the locations on a substrate that are exposed to light. Some systems and methods useful in the design and/or use of such masks are described in the following U.S. Pat. Nos. 5,571,639 to Hubbell, et al.; 5,593,839 to Hubbell, et al.; 5,856,101 to Hubbell, et al.; 6,153,743 to Hubbell, et al.; and 6,188,783 to Balaban, et al., each of which is hereby incorporated herein by reference for all purposes.

The present invention relates to release of polymers, including nucleic acid probes, from an array.

### SUMMARY OF THE INVENTION

Methods are provided for releasing polymers from an array of polymers having the steps of providing a substrate; attaching a linker comprising a releasable group to the substrate, wherein the releasable group is labile under a set of conditions; attaching a first monomer to the linker; attaching a second monomer to the linker or to the first monomer repeating the step of attaching a second monomer until a polymer is synthesized; and releasing the polymer using the set of conditions. Arrays of releasable polymers are provided, the array comprising a substrate having a linker comprising a releasable group which is labile under a set of conditions and attached to said linker a polymer, wherein the polymer can be released by exposure of the array to the set of conditions.

The present invention also discloses nucleic acid arrays having a releasable nucleic acid probe, the nucleic acid array comprising a substrate having attached thereto a nucleic acid probe, the nucleic acid probe comprising a releasable group which is labile under a set of conditions wherein the releasable group allows release of the probe upon activation. Also provided are methods for fabricating a polymer array having releasable polymers, the method having the steps of: providing a substrate; attaching a linker to the substrate, the linker comprising a releasable group which is labile under a set of conditions; reversibly modifying the releasable group with a protecting group to provide a reversibly modified releasable group wherein the modified releasable group is not labile under the set of conditions; attaching a first monomer to the linker; attaching a second monomer to the linker or to the first monomer; repeating the step of attaching the second monomer until a polymer is provided; and demodifying the reversibly modified releasable group.

### DETAILED DESCRIPTION OF THE INVENTION

The present invention has many preferred embodiments and relies on many patents, applications and other references for details known to those of the art. Therefore, when a patent, application, or other reference is cited or repeated below, it should be understood that it is incorporated by reference in its entirety for all purposes as well as for the proposition that is recited.

The following definitions are set forth to illustrate and define the meaning and scope of the various terms used to describe the invention herein.

The term “alkyl” refers to a branched or straight chain acyclic, monovalent saturated hydrocarbon radical of one to twenty carbon atoms.

The term “alkenyl” refers to an unsaturated hydrocarbon radical which contains at least one carbon-carbon double bond and includes straight chain, branched chain and cyclic radicals.

The term “alkynyl” refers to an unsaturated hydrocarbon radical which contains at least one carbon-carbon triple bond and includes straight chain, branched chain and cyclic radicals.

The term “aryl” refers to an aromatic monovalent carbocyclic radical having a single ring (e.g., phenyl) or two condensed rings (e.g., naphthyl), which can optionally be mono-, di-, or tri-substituted, independently, with alkyl, lower-alkyl, cycloalkyl, hydroxylower-alkyl, aminolower-alkyl, hydroxyl, thiol, amino, halo, nitro, lower-alkylthio, lower-alkoxy, mono-lower-alkylamino, di-lower-alkylamino, acyl, hydroxycarbonyl, lower-alkoxycarbonyl, hydroxysulfonyl, lower-alkoxysulfonyl, lower-alkylsulfonyl, lower-alkylsulfinyl, trifluoromethyl, cyano, tetrazoyl, carbamoyl, lower-alkylcarbamoyl, and di-lower-alkylcarbamoyl. Alternatively, two adjacent positions of the aromatic ring may be substituted with a methylenedioxy or ethylenedioxy group.

The term “heteroaromatic” refers to an aromatic monovalent mono- or poly-cyclic radical having at least one heteroatom within the ring, e.g., nitrogen, oxygen or sulfur, wherein the aromatic ring can optionally be mono-, di- or tri-substituted, independently, with alkyl, lower-alkyl, cycloalkyl, hydroxylower-alkyl, aminolower-alkyl, hydroxyl, thiol, amino, halo, nitro, lower-alkylthio, lower-alkoxy, mono-lower-alkylamino, di-lower-alkylamino, acyl, hydroxycarbonyl, lower-alkoxycarbonyl, hydroxysulfonyl, lower-alkoxysulfonyl, lower-alkylsulfonyl, lower-alkylsulfinyl, trifluoromethyl, cyano, tetrazoyl, carbamoyl, lower-alkylcarbamoyl, and di-lower-alkylcarbamoyl. For example, typical heteroaryl groups with one or more nitrogen atoms are tetrazoyl, pyridyl (e.g., 4-pyridyl, 3-pyridyl, 2-pyridyl), pyrrolyl (e.g., 2-pyrrolyl, 2-(N-alkyl)pyrrolyl), pyridazinyl, quinolyl (e.g. 2-quinolyl, 3-quinolyl etc.), imidazolyl, isoquinolyl, pyrazolyl, pyrazinyl, pyrimidinyl, pyridonyl or pyridazinonyl; typical oxygen heteroaryl radicals with an oxygen atom are 2-furyl, 3-furyl or benzofuranyl; typical sulfur heteroaryl radicals are thienyl, and benzothienyl; typical mixed heteroatom heteroaryl radicals are furazanyl and phenothiazinyl. Further the term also includes instances where a heteroatom within the ring has been oxidized, such as, for example, to form an N-oxide or sulfone.

The term “optionally substituted” refers to the presence or lack thereof of a substituent on the group being defined. When substitution is present the group may be mono-, di- or tri-substituted, independently, with alkyl, lower-alkyl, cycloalkyl, hydroxylower-alkyl, aminolower-alkyl, hydroxyl, thiol, amino, halo, nitro, lower-alkylthio, lower-alkoxy, mono-lower-alkylamino, di-lower-alkylamino, acyl, hydroxycarbonyl, lower-alkoxycarbonyl, hydroxysulfonyl, lower-alkoxysulfonyl, lower-alkylsulfonyl, lower-alkylsulfinyl, trifluoromethyl, cyano, tetrazoyl, carbamoyl, lower-alkylcarbamoyl, and di-lower-alkylcarbamoyl. Typically, electron-donating substituents such as alkyl, lower-alkyl, cycloalkyl, hydroxylower-alkyl, aminolower-alkyl, hydroxyl, thiol, amino, halo, lower-alkylthio, lower-alkoxy, mono-lower-alkylamino and di-lower-alkylamino are preferred.

The term “electron donating group” refers to a radical group that has a lesser affinity for electrons than a hydrogen atom would if it occupied the same position in the molecule. For example, typical electron donating groups are hydroxy, alkoxy (e.g. methoxy), amino, alkylamino and dialkylamine.

The term “leaving group” means a group capable of being displaced by a nucleophile in a chemical reaction, for example halo, nitrophenoxy, pentafluorophenoxy, alkyl sulfonates (e.g., methanesulfonate), aryl sulfonates, phosphates, sulfonic acid, sulfonic acid salts, and the like.

“Activating group” refers to those groups which, when attached to a particular functional group or reactive site, render that site more reactive toward covalent bond formation with a second functional group or reactive site. The group of activating groups which are useful for a carboxylic acid include simple ester groups and anhydrides. The ester groups include alkyl, aryl and alkenyl esters and in particular such groups as 4-nitrophenyl, N-hydroxylsuccinimide and pentafluorophenol. Other activating groups are known to those of skill in the art.

“Chemical library” or “array” is an intentionally created collection of differing molecules which can be prepared either synthetically or biosynthetically and screened for activity in a variety of different formats (e.g., libraries of soluble molecules; and libraries of compounds tethered to resin beads, silica chips, or other solid supports). The term is also intended to refer to an intentionally created collection of stereoisomers.

“Predefined region” refers to a localized area on a solid support. It can be where synthesis takes place or where a nucleic acid is placed. Predefined region can also be defined as a “selected region.” The predefined region may have any convenient shape, e.g., circular, rectangular, elliptical, wedge-shaped, etc. For the sake of brevity herein, “predefined regions” are sometimes referred to simply as “regions.” In some embodiments, a predefined region and, therefore, the area upon which each distinct compound is synthesized or placed is smaller than about 1 cm² or less than 1 mm². Within these regions, the molecule therein is preferably in a substantially pure form. In additional embodiments, a predefined region can be achieved by physically separating the regions (i.e., beads, resins, gels, etc.) into wells, trays, etc.

A “linker” is a molecule or group of molecules attached to a substrate and spacing a synthesized polymer from the substrate for exposure/binding to a receptor.

“Solid support”, “support”, and “substrate” refer to a material or group of materials having a rigid or semi-rigid surface or surfaces. In many embodiments, at least one surface of the solid support will be substantially flat, although in some embodiments it may be desirable to physically separate synthesis regions for different compounds with, for example, wells, raised regions, pins, etched trenches, or the like. According to other embodiments, the solid support(s) will take the form of beads, resins, gels, microspheres, or other geometric configurations.

Isolation and purification of the compounds and intermediates described herein can be effected, if desired, by any suitable separation or purification procedure such as, for example, filtration, extraction, crystallization, column chromatography, thin-layer chromatography, thick-layer (preparative) chromatography, distillation, or a combination of these procedures.

A “channel block” is a material having a plurality of grooves or recessed regions on a surface thereof. The grooves or recessed regions may take on a variety of geometric configurations, including but not limited to stripes, circles, serpentine paths, or the like. Channel blocks may be prepared in a variety of manners, including etching silicon blocks, molding or pressing polymers, etc.

A “monomer” is a member of the set of small molecules which can be joined together to form a polymer. The set of monomers includes but is not restricted to, for example, the set of common L-amino acids, the set of common D-amino acids, the set of synthetic amino acids, the set of nucleotides and the set of pentoses and hexoses. As used herein, monomer refers to any member of a basis set for synthesis of a polymer. Thus, monomers refers to dimmers, trimers, tetramers and higher units of molecules which can be joined to form a polymer. For example, dimmers of the 20 naturally occurring L-amino acids for a basis set of 400 monomers for synthesis of polypeptides. Different basis sets of monomers may be used at successive steps in the synthesis of a polymer. Furthermore, each of the sets may include protected members which are modified after synthesis.

A “polymer” is composed of two or more joined monomers and includes for example both linear and cyclic polymers of nucleic acids, polysaccharides, phospholipids, and peptides having either α-, β-, and ω-amino acids, hetero-polymers in which a known drug is covalently bound to any of the above, polyurethanes, polyesters, polycarbonates, polyureas, polyamides, polyethyleneimines, polyarylene sulfides, polysiloxanes, polyimides, polyacetates, or other polymers.

A “releasable group” is a moiety or chemical group which is labile, i.e., may be activated or cleaved, under a given set of conditions, but is stable under other sets of conditions.

As used in this application, the singular form “a,” “an,” and “the” include plural references unless the context clearly dictates otherwise. For example, the term “an agent” includes a plurality of agents, including mixtures thereof.

An individual is not limited to a human being but may also be other organisms including but not limited to mammals, plants, bacteria, or cells derived from any of the above.

Throughout this disclosure, various aspects of this invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

The practice of the present invention may employ, unless otherwise indicated, conventional techniques and descriptions of organic chemistry, polymer technology, molecular biology (including recombinant techniques), cell biology, biochemistry, and immunology, which are within the skill of the art. Such conventional techniques include polymer array synthesis, hybridization, ligation, and detection of hybridization using a label. Specific illustrations of suitable techniques can be had by reference to the example herein below. However, other equivalent conventional procedures can, of course, also be used. Such conventional techniques and descriptions can be found in standard laboratory manuals such as Genome Analysis: A Laboratory Manual Series (Vols. I-IV), Using Antibodies: A Laboratory Manual, Cells: A Laboratory Manual, PCR Primer: A Laboratory Manual, and Molecular Cloning: A Laboratory Manual (all from Cold Spring Harbor Laboratory Press), Stryer, L. (1995) Biochemistry (4th Ed.) Freeman, New York, Gait, “Oligonucleotide Synthesis: A Practical Approach” 1984, IRL Press, London, Nelson and Cox (2000), Lehninger, Principles of Biochemistry 3^{rd} Ed., W.H. Freeman Pub., New York, N.Y. and Berg et al. (2002) Biochemistry, 5^{th} Ed., W.H. Freeman Pub., New York, N.Y., all of which are herein incorporated in their entirety by reference for all purposes.

The present invention can employ solid substrates, including arrays in some preferred embodiments. Methods and techniques applicable to polymer (including protein) array synthesis have been described in U.S. Ser. No. 09/536,841, WO 00/58516, U.S. Pat. Nos. 5,143,854, 5,242,974, 5,252,743, 5,324,633, 5,384,261, 5,405,783, 5,424,186, 5,451,683, 5,482,867, 5,491,074, 5,527,681, 5,550,215, 5,571,639, 5,578,832, 5,593,839, 5,599,695, 5,624,711, 5,631,734, 5,795,716, 5,831,070, 5,837,832, 5,856,101, 5,858,659, 5,936,324, 5,968,740, 5,974,164, 5,981,185, 5,981,956, 6,025,601, 6,033,860, 6,040,193, 6,090,555, 6,136,269, 6,269,846 and 6,428,752, in PCT Applications Nos. PCT/US99/00730 (International Publication Number WO 99/36760) and PCT/US01/04285, which are all incorporated herein by reference in their entirety for all purposes.

Patents that describe synthesis techniques in specific embodiments include U.S. Pat. Nos. 5,412,087, 6,147,205, 6,262,216, 6,310,189, 5,889,165, and 5,959,098. Nucleic acid arrays are described in many of the above patents, but the same techniques are applied to polypeptide arrays.

Nucleic acid arrays that are useful in the present invention include those that are commercially available from Affymetrix (Santa Clara, Calif.) under the brand name GeneChip®. Example arrays are shown on the website at affymetrix.com.

The present invention also contemplates many uses for polymers attached to solid substrates. These uses include gene expression monitoring, profiling, library screening, genotyping and diagnostics. Gene expression monitoring, and profiling methods can be shown in U.S. Pat. Nos. 5,800,992, 6,013,449, 6,020,135, 6,033,860, 6,040,138, 6,177,248 and 6,309,822. Genotyping and uses therefore are shown in U.S. Ser. No. 60/319,253, 10/013,598, and U.S. Pat. Nos. 5,856,092, 6,300,063, 5,858,659, 6,284,460, 6,361,947, 6,368,799 and 6,333,179. Other uses are embodied in U.S. Pat. Nos. 5,871,928, 5,902,723, 6,045,996, 5,541,061, and 6,197,506.

The present invention also contemplates sample preparation methods in certain preferred embodiments. Prior to or concurrent with genotyping, the genomic sample may be amplified by a variety of mechanisms, some of which may employ PCR. See, e.g., PCR Technology: Principles and Applications for DNA Amplification (Ed. H. A. Erlich, Freeman Press, NY, N.Y., 1992); PCR Protocols: A Guide to Methods and Applications (Eds. Innis, et al., Academic Press, San Diego, Calif., 1990); Mattila et al., Nucleic Acids Res. 19, 4967 (1991); Eckert et al., PCR Methods and Applications 1, 17 (1991); PCR (Eds. McPherson et al., IRL Press, Oxford); and U.S. Pat. Nos. 4,683,202, 4,683,195, 4,800,159 4,965,188,and 5,333,675, and each of which is incorporated herein by reference in their entireties for all purposes. The sample may be amplified on the array. See, for example, U.S. Pat. No. 6,300,070 and U.S. patent application Ser. No. 09/513,300, which are incorporated herein by reference.

Other suitable amplification methods include the ligase chain reaction (LCR) (e.g., Wu and Wallace, Genomics 4, 560 (1989), Landegren et al., Science 241, 1077 (1988) and Barringer et al. Gene 89:117 (1990)), transcription amplification (Kwoh et al., Proc. Natl. Acad. Sci. USA 86, 1173 (1989) and WO88/10315), self-sustained sequence replication (Guatelli et al., Proc. Nat. Acad. Sci. USA, 87, 1874 (1990) and WO90/06995), selective amplification of target polynucleotide sequences (U.S. Pat. No. 6,410,276), consensus sequence primed polymerase chain reaction (CP-PCR) (U.S. Pat. No. 4,437,975), arbitrarily primed polymerase chain reaction (AP-PCR) (U.S. Pat. Nos. 5,413,909, 5,861,245) and nucleic acid based sequence amplification (NABSA). (See, U.S. Pat. Nos. 5,409,818, 5,554,517, and 6,063,603, each of which is incorporated herein by reference). Other amplification methods that may be used are described in, U.S. Pat. Nos. 5,242,794, 5,494,810, 4,988,617 and in U.S. Ser. No. 09/854,317, each of which is incorporated herein by reference.

Additional methods of sample preparation and techniques for reducing the complexity of a nucleic sample are described in Dong et al., Genome Research 11, 1418 (2001), in U.S. Pat. Nos. 6,361,947, 6,391,592 and U.S. patent application Ser. Nos. 09/916,135, 09/920,491, 09/910,292, and 10/013,598.

Methods for conducting polynucleotide hybridization assays have been well developed in the art. Hybridization assay procedures and conditions will vary depending on the application and are selected in accordance with the general binding methods known including those referred to in: Maniatis et al. Molecular Cloning: A Laboratory Manual (2^{nd} Ed. Cold Spring Harbor, N.Y, 1989); Berger and Kimmel Methods in Enzymology, Vol. 152, Guide to Molecular Cloning Techniques (Academic Press, Inc., San Diego, Calif., 1987); Young and Davism, P.N.A.S, 80: 1194 (1983). Methods and apparatus for carrying out repeated and controlled hybridization reactions have been described in U.S. Pat. Nos. 5,871,928, 5,874,219, 6,045,996 and 6,386,749, 6,391,623 each of which are incorporated herein by reference

The present invention also contemplates signal detection of hybridization between ligands in certain preferred embodiments. See U.S. Pat. Nos. 5,143,854, 5,578,832; 5,631,734; 5,834,758; 5,936,324; 5,981,956; 6,025,601; 6,141,096; 6,185,030; 6,201,639; 6,218,803; and 6,225,625, in U.S. Patent application 60/364,731 and in PCT Application PCT/US99/06097 (published as WO99/47964), each of which also is hereby incorporated by reference in its entirety for all purposes.

Methods and apparatus for signal detection and processing of intensity data are disclosed in, for example, U.S. Pat. Nos. 5,143,854, 5,547,839, 5,578,832, 5,631,734, 5,800,992, 5,834,758; 5,856,092, 5,902,723, 5,936,324, 5,981,956, 6,025,601, 6,090,555, 6,141,096, 6,185,030, 6,201,639; 6,218,803; and 6,225,625, in U.S. Patent application 60/364,731 and in PCT Application PCT/US99/06097 (published as WO99/47964), each of which also is hereby incorporated by reference in its entirety for all purposes.

The practice of the present invention may also employ conventional biology methods, software and systems. Computer software products of the invention typically include computer readable medium having computer-executable instructions for performing the logic steps of the method of the invention. Suitable computer readable medium include floppy disk, CD-ROM/DVD/DVD-ROM, hard-disk drive, flash memory, ROM/RAM, magnetic tapes and etc. The computer executable instructions may be written in a suitable computer language or combination of several languages. Basic computational biology methods are described in, e.g. Setubal and Meidanis et al., Introduction to Computational Biology Methods (PWS Publishing Company, Boston, 1997); Salzberg, Searles, Kasif, (Ed.), Computational Methods in Molecular Biology, (Elsevier, Amsterdam, 1998); Rashidi and Buehler, Bioinformatics Basics: Application in Biological Science and Medicine (CRC Press, London, 2000) and Ouelette and Bzevanis Bioinformatics: A Practical Guide for Analysis of Gene and Proteins (Wiley & Sons, Inc., 2^{nd} ed., 2001).

The present invention may also make use of various computer program products and software for a variety of purposes, such as probe design, management of data, analysis, and instrument operation. See, U.S. Pat. Nos. 5,593,839, 5,795,716, 5,733,729, 5,974,164, 6,066,454, 6,090,555, 6,185,561, 6,188,783, 6,223,127, 6,229,911 and 6,308,170.

Additionally, the present invention may have preferred embodiments that include methods for providing genetic information over networks such as the Internet as shown in U.S. patent applications Nos. 10/063,559, 60/349,546, 60/376,003, 60/394,574, 60/403,381.

In one aspect of the present invention, a method is provided for releasing polymers from an array of polymers having the steps of providing a substrate; attaching a linker comprising a releasable group to the substrate, wherein the releasable group is labile under a set of conditions; attaching a first monomer to the linker; attaching a second monomer to the linker or to the first monomer; repeating the step of attaching a second monomer until a polymer is synthesized; and releasing the polymer using the set of conditions.

In a preferred embodiment of the present invention, the monomers are nucleotides or amino acids. In a preferred embodiment of the present invention, the releasable group is a photogroup. The photogroup is preferably activated by light having a wavelength of 313 nm and below.

In on aspect of the present invention, a releasable polymer array is provided having a substrate having a linker comprising a releasable group which is labile under a set of conditions and attached to the linker a polymer, wherein the polymer can be released by exposure of the array to the set of conditions.

In preferred embodiments of the present invention, the polymer is a nucleic acid or peptide. Most preferably, the polymer is an oligonucleotide. Preferably, the releasable group is a photogroup.

In one aspect of the present invention, a nucleic acid array is presented having a releasable nucleic acid probe, the nucleic acid array having a substrate having attached thereto a nucleic acid probe, the nucleic acid probe comprising a releasable group which is labile under a set of conditions wherein the releasable group allows release of the probe upon activation. In one preferred embodiment, the releasable group comprises a photogroup.

In one aspect of the present invention, a method for fabricating a polymer array having releasable polymers is presented, the method having the following steps (in no particular order): providing a substrate; attaching a linker to the substrate, the linker comprising a releasable group which is labile under a set of conditions; reversibly modifying the releasable group with a protecting group to provide a reversibly modified releasable group wherein the modified releasable group is not labile under the set of conditions; attaching a first monomer to the linker; attaching a second monomer to the linker or to the first monomer; repeating the step of attaching the second monomer until a polymer is provided; and demodifying the reversibly modified releasable group. In one preferred embodiment of the present invention, the releasable group comprises a photogroup.

In accordance with the present invention, conditions or sets of conditions which may be used to activate a releasable group depend upon the chemical nature of the moiety. Thus, releasable groups containing photogroups may be activated or cleaved using the appropriate wavelength of electromagnetic radiation. The releasable group, depending about its chemical nature, may alternatively be an electrochemically-sensitive group which may be cleaved in the presence of an electric field or an electric current. In still further alternative embodiments, ion beams, electron beams, or the like may be used to cleave the releasable group. In accordance with one aspect of the present invention, releasable groups may be used in conjunction with capture probes as described in U.S. application Ser. No. 10/272,155 filed on Oct. 14, 2002, incorporated here by referenced in its entirety.

With regard to the use of an electric field to activate a releasable group, alcohol groups, such as those found in nucleosides used in oligonucleotide synthesis, can for example be protected with a benzoate ester which can be electrolytically reduced to cleave the benzoate ester and reform the alcohol (Greene, et al., Protective Groups in Organic Synthesis (1991) (incorporated here by reference). Amine groups, for example, such as those found in amino acids used for protein synthesis, can be protected with a benzyl carbamate group which can be electrolytically reduced to regenerate the amino groups. (Greene, et al.).

In one preferred embodiment of the present invention, nucleic acid probes may be released from a solid support by virtue of a releasable group through which the nucleic acid probe is connected to the solid support. In accordance with this aspect of the present invention, a releasable group must be substantially stable under the conditions used to attach the nucleic acid in question to the support, but labile, i.e., cleavable or activatable, under other conditions which are not employed to attach the nucleic acid to the solid support. The releasable group is preferably employed at the base or terminus of a nucleic acid probe to attach the probe to the solid surface such that the entire nucleic acid probe can be released upon activation or cleavage of the releasable group. Alternatively, a predetermined part of an oligonucleotide probe may be released by placement of the releasable group in positions other than the base of the probe. In accordance with the present invention, the releasable group may be attached to the nucleic acid probe at either the 5′ or 3′ ends.

In accordance with one aspect of the present invention, linker molecules are provided on a substrate having a surface. One end of the linker molecule is located away from the surface and another is attached to the substrate. The terminal end of the linker molecule situated away from the substrate is provided with a reactive functional group protected with a photoremovable protective group, which is removable at a first wavelength of light. The linker also has a releasable group which is activatable with a second wavelength of light, which is different than the first wavelength of light, or alternatively with an electric field, but where the releasable group is substantially stable at the first wavelength of light or in the absence of an electric field, the releasable groups situated in or on the linker in such a manner that the reactive functional group (or anything subsequently attached to it) is detached from the linker upon activation of the releasable group.

Using lithographic methods, the photoremovable protective group is exposed to light and removed from the linker molecules in first selected regions. As the releasable group is stable, or at least substantially stable, under these conditions it remains intact. The substrate is then washed or otherwise contacted with a first monomer which also bears the photoremovable protective group, which reacts with the exposed functional groups on the linker molecules, yielding a linker molecule, terminating in a monomer bearing the photoremovable protective group. In preferred embodiments, the monomer is an amino acid containing the photoremovable protecting group at its amino or carboxy terminus and the linker molecule terminates in an amino or carboxy acid group bearing a photoremovable protecting group. In another preferred embodiment, the monomer is a nucleotide containing the photoremovable protecting group at its 5′ or 3′ end and the linker molecule terminates in a 5′ or 3′ nucleotide bearing the photoremovable protecting group. Photoremovable protecting groups which might be employed with respect to one aspect of the present invention include methyl-6-nitropiperonyloxycarbonyl (MeNPOC), 6-nitrobenzyloxycarbonyl group (NBOC), or 6-nitroveratryloxycarbonyl group (NVOC) or derivatives or variants thereof.

A second set of selected regions is, thereafter, exposed to light and the photoremovable protective group on the linker molecule or monomer is removed at the second set of regions to expose functional groups. The substrate is then contacted with a second monomer for reaction with exposed functional groups. This process is repeated to selectively apply monomers until polymers of a desired length and desired chemical sequence are obtained.

In accordance with one aspect of the present invention, after fabrication of the polymers on the surface of the substrate as described above, the array may be exposed to conditions which activate the releasable group, releasing the polymer from the surface of the array. Releasing the polymer from the array may be done immediately after fabrication of the polymers is complete, i.e. before any further use is made of the array. Alternatively, the releasable polymer array may first be used for an application prior to release of the polymers. For example, where the polymers are oligonucleotides, the releasable oligonucleotide array may be used for nucleic acid analysis, including hybridization to samples of DNA or RNA prior to release. Subsequently, in accordance with one aspect of the present invention, the oligonucleotide probe, which may be hybridized to another nucleic acid, may be released from the surface of the array via activation or cleavage of the releasable group. Further experimentation, such as for example sequencing, cloning, hybridization, amplification, etc., may then be performed with the released nucleic acid.

In a preferred embodiment of the present invention, photolithography is used to fabricate a releasable array of nucleic acid probes. In accordance with this aspect of the present invention, the releasable group must be substantially stable under the conditions employed in the photolithographic process, including the wavelengths of light used to deprotect the growing chains of oligonucleotides, but cleavable under other conditions not used to fabricate the array. In accordance with this aspect of the present invention, a releasable group which is activated at a shorter wavelength of radiation or light, but is stable under the longer wavelengths used in photolithography is preferred.

Herein, radiation means energy which may be selectively applied including energy having a wavelength of between 10⁻¹⁴ and 10⁴ meters including, for example, electron beam radiation, gamma radiation, x-ray radiation, ultra-violet radiation, visible light, infrared radiation, microwave radiation, and radio waves. “Irradiation” refers to the application of radiation to a surface. In accordance with one aspect of the present invention, the term light may be used to refer to all portions of the electromagnetic spectrum.

The wavelength of radiation to be employed in cleaving a releasable group containing a photogroup or moiety, in accordance with one aspect of the present invention, may be determined by determining the wavelength of light which activates the photogroup. For example, if the photo moiety is activated by ultra-violet radiation of 313 nm, light of 313 nm would be used to cleave the releasable group. The wavelength of light at which a photo moiety is activated may be determined from the literature or experimentally from techniques know to those of skill in the art.

In accordance with one aspect of the present invention, it is preferred that photo moieties employed in a releasable group are activatable at wavelengths of radiation other than 365 nm. In this regard, one photolithographic process used to produce arrays employs photoremovable protecting groups for protection of functional groups, such as hydroxyl groups, that are activated at or around 365 nm. See, e.g., U.S. Pat. No. 6,261,776, incorporated here in its entirety by reference. In a preferred embodiment of the present invention, releasable groups have a very limited activation, preferably none, at 365 nm. In accordance with one aspect of the present invention, photogroups may be identified having substantially no absorbance at 365 nm, but which absorb at shorter wavelengths. Preferably, according to one aspect of the present invention, releasable groups are activated at 313 nm and below.

In accordance with one aspect of the present invention, the conditions under which a releasable group is activatable are modified or changed through reversible modification of the group to provide a reversibly protected releasable group. The reversibly protected releasable group is not activated under the conditions the unmodified releasable group could be activated at. However, in accordance with this aspect of the present invention, the reversibly protected releasable group may be rendered activatable under its normal set of conditions by reversing the modification which rendered the group non-activatable.

In a preferred embodiment of this aspect of the present invention, the releasable group is a photogroup or moiety. In accordance with the present invention, the photogroup is reversibly modified such that it is protected from photo activation at its normal activation wavelength of light. With respect to this aspect of the present invention, the photogroup can be demodified to provide a releasable group which may be activated at the photogroups normal activation wavelength.

In one aspect of the present invention, linker molecules are provided on a substrate having a surface. One end of the linker molecule is located away from the surface and another is attached to the surface of the substrate. The terminal end of the linker molecule situated away from the substrate is provided with a reactive functional group protected with a photoremovable protective group, which is removable at a wavelength of light. The linker also has a releasable group, situated in or on the linker in such a manner that the reactive functional group (or anything subsequently attached to it) is detached from the linker upon activation of the releasable group, the releasable group comprising a photogroup which is activatable with the wavelength of light. In accordance with this aspect of the present invention, the releasable group is reversibly modified to provide a reversibly protected photogroup which is substantially stable at the wavelength of light. The reversible modification of the photogroup in the releasable group may be performed at any time in accordance with the present invention. Thus, the photogroup may be modified either before or after the linker is attached to the substrate.

Using lithographic methods, the photoremovable protective group is exposed to light and removed from the linker molecules in first selected regions. The substrate is then washed or otherwise contacted with a first monomer, bearing the photoremovable protective group, that reacts with the exposed functional groups on the linker molecules, yielding a linker molecule, terminating in a monomer bearing the photoremovable protective group. In one preferred embodiment, the monomer is an amino acid containing the photoremovable protecting group at its amino or carboxy terminus and the linker molecule terminates in an amino or carboxy acid group bearing a photoremovable protecting group. In another preferred embodiment, the monomer is a nucleotide containing the photoremovable protecting group at its 5′ or 3′ end and the linker molecule terminates in a 5′ or 3′ nucleotide bearing the photoremovable protecting group. Preferably, photoremoval protecting groups which may be employed with respect to one aspect of the present invention include methyl-6-nitropiperonyloxycarbonyl (MeNPOC), 6-nitrobenzyloxycarbonyl group (NBOC), or 6-nitroveratryloxycarbonyl group (NVOC) or derivatives or variants thereof as appropriate.

A second set of selected regions is, thereafter, exposed to light and the photoremovable protective group on the linker molecule or monomer is removed at the second set of regions. The substrate is then contacted with a second monomer for reaction with exposed functional groups. This process is repeated to selectively apply monomers until polymers of a desired length and desired chemical sequence are obtained.

In accordance with one aspect of the present invention, after fabrication of the polymers on the surface of the substrate as described above, the reversibly protected releasable group is exposed to conditions which reverse the modification to the releasable group. The array may then be exposed to conditions which activate the releasable group, releasing the polymer from the surface of the array. Activation of the release group may be performed either before or after the array has been used in an application. In a preferred embodiment of the present invention, the monomers are nucleotides and the polymers are oligonucleotides. This oligonucleotide array may be used for nucleic acid analysis, including hybridization to samples of DNA or RNA. Subsequently, in accordance with one preferred embodiment, the oligonucleotide probe, which may be hybridized to another nucleic acid, may be released from the surface of the array via activation or cleavage of the releasable group as set forth above. Further experimentation, such as for example sequencing, cloning, hybridization, amplification, etc., may then be performed with the released nucleic acid.

In accordance with this aspect of the present invention, standard photo protecting groups such as MeNPOC, NBOC, or NVOC may be incorporated into a releasable group and reversibly modified to provide protected MeNPOC, NBOC, or NVOC groups to prevent their normal photoactivation at or around 365 nm. In accordance with this aspect of the present invention, the same photogroup may be used in the releasable group as is employed to protect the hydroxyl groups of the growing oligonucleotide chain.

The appropriate demodification chemical environment may be determined by a person of ordinary skill based on the disclosures herein and the chemistry of the group used to modify or protect the releasable group. In accordance with this aspect of the present invention, it is important that the demodification chemical environment does not adversely affect the nucleic acid array. Suitable conditions for demodification may be determined by those of ordinary skill based on the disclosures herein, the chemistry of the protecting group used to modify the releasable group and the stabilities of the various bonds in the polymer array under different chemical conditions.

In some embodiments of the present invention, a plurality of different releasable groups or reversibly protected releasable groups may be employed on a polymer array such that predetermined polymers may be released by chosen conditions. For example, in accordance with this aspect of the present invention, a plurality of different releasable groups, comprising photogroups, having different patterns of photoactivation, may be employed at predetermined locations of a nucleic acid array to allow release of preselected nucleic acid probes at different wavelengths of light. Alternatively, an electrically activated releasable group may be provided in some locations of an array and a releasable group comprising a photogroup may be employed in others to provide for selective release of polymers on the surface of the array.

### EXAMPLES

In accordance with the present invention, it is preferred that the releasable group comprise a photogroup which is selected from the set forth below:

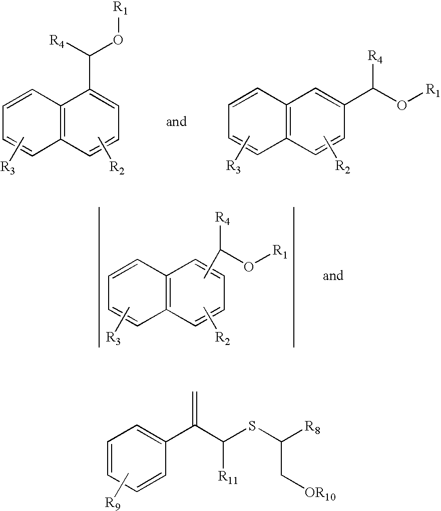

In accordance with the present invention, it is preferred that a releasable group be modified with a chemical moiety of the structure:

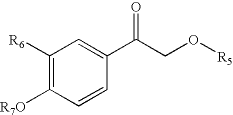

In accordance with this aspect of the present invention, the groups disclosed above may be functionalized to be used in a DNA synthesizer as DMT/phosphoramidite derivatives.

As used above, R₁, R₅ and R₁₀ are, independently, a DMT group (4,4′dimethoxytrityl), a carbonate, or a phosphate. R₄, R₈ and R₁₁ are, independently H, alkly, alkenyl, or substituted aryl. R₂, R₃, R₆, and R₉ are, independently, H, or a substituted alkoxy, alkyl, alkenyl, aryl, amine or carboxcylic acid. R7 is a substituted silyl group.

Reference literature on the mechanism of photolysis include the following references: DeCosta, D. P. and Pincock, J. A. J.Am.Chem.Soc. 1989, 111, 8948-8950; DeCosta, D. P. and Pincock, J. A. J.Am.Chem.Soc. 1993, 115, 2180-2190; and Givens R. S. and Matuszewski B., J. Am.Chem.Soc. 1984, 6860-6861.

Styryl thioethers compounds are described, inter alia, in Fox, M. A. and Tribel, C. A. J.Org.Chem. 1983, 48, 835-840. p-hydroxyphenacil compounds are described, inter alia, in Zhang, L., Corrie, J. E. T., Ranjit, V., Munashinghe, N., Wan, P., J.Am.Chem.Soc. 1999, 121, 5625-5632.

Each of the above references is incorporated here by reference for all purposes.

What is claimed is:

1. A method for releasing polymers from an array of polymers comprising the steps of
providing a substrate;
attaching a linker comprising a releasable group to the substrate, wherein said releasable group is labile under a set of conditions;
attaching a first monomer to the linker;
attaching a second monomer to the linker or to the first monomer
repeating said step of attaching a second monomer until a polymer is synthesized; and
releasing said polymer using the set of conditions.

2. The method of claim 1 wherein said monomers are nucleotides.

3. The method of claim 1 wherein said releasable group comprises a photogroup.

4. The method of claim 3 wherein said photogroup is activated by light having a wavelength of 313 nm and below.

5. The method of claim 1 wherein said monomers are amino acids.

6. A releasable polymer array comprising a substrate having a linker comprising a releasable group which is labile under a set of conditions and attached to said linker a polymer, wherein said polymer can be released by exposure of the array to the set of conditions.

7. A releasable polymer array according to claim 6 wherein said polymer is a nucleic acid.

8. A releasable polymer array according to claim 7 wherein said nucleic acid is an oligonucleotide.

9. A releasable polymer array according to claim 6 wherein said releasable group comprises a photogroup.

10. A releasable polymer array according to claim 6 wherein said polymer is a peptide.

11. A nucleic acid array having a releasable nucleic acid probe, said nucleic acid array comprising a substrate having attached thereto a nucleic acid probe, said nucleic acid probe comprising a releasable group which is labile under a set of conditions wherein said releasable group allows release of the probe upon activation.

12. A nucleic acid array according to claim 11 wherein said releasable group comprises a photogroup which may be activated by light having a wavelength of 313 nm and below.

13. A method for fabricating a polymer array having releasable polymers, said method comprising the steps of:
providing a substrate;
attaching a linker to said substrate, said linker comprising a releasable group which is labile under a set of conditions;
reversibly modifying said releasable group with a protecting group to provide a reversibly modified releasable group wherein said modified releasable group is not labile under the set of conditions;
attaching a first monomer to said linker;
attaching a second monomer to said linker or to the first monomer;
repeating said step of attaching said second monomer until a polymer is provided; and
demodifying said reversibly modified releasable group.

14. A method for fabricating a polymer array according to claim 13 wherein said releasable group comprises a photogroup.
